(19) United States
(12) Patent Application Publication
(10) Pub. No.: US 2008/0103295 A1
(43) Pub. Date: May 1, 2008

(54) PROCESS FOR THE PREPARATION OF SUCROSE-6-ESTER BY ESTERIFICATION IN THE PRESENCE OF SOLID SUPERACID CATALYST

(76) Inventors: David Losan Ho, Los Angeles, CA (US); Wan Zhenghao, Wyuishan City (CN)

Correspondence Address: PERKINS COIE LLP, POST OFFICE BOX 1208, SEATTLE, WA, 98111-1208, US

(21) Appl. No.: 11/552,813

(22) Filed: Oct. 25, 2006

(51) Int. Cl. C07H 15/00; C13K 13/00

(52) U.S. Cl. 536/18.4; 536/123.13

(57) ABSTRACT

One embodiment of the present invention is a process of making sucrose-6-ester from sucrose by transesterification in the presence of a solid super acid catalyst such as SO₄²⁻—TiO₂/Al₂O₃ or SO₄²⁻—TiO₂. The sucrose-6-acetate is then chlorinated to afford sucralose-6-acetate, using BTC or thionyl chloride. Sucralose-6-acetate is converted into TPSGA for the purpose of purification. TPSGA is de-esterified by sodium methoxide/methanol or sodium ethoxide/ethanol to give sucralose.

### FIELD OF THE INVENTION

The present invention relates methods of making sucralose.

### BACKGROUND OF THE INVENTION

Sucralose (4,1′,6′-chloro-4,1′,6′-trideoxygalactosucrose) is a non-calorie sweetner that is produced by selective chlorination of sucrose. It is 400-600 times as sweet as sucrose and provides a clean sweet taste that does not leave an unpleasant aftertaste. Its exceptional heat stability makes sucralose a promising sugar substitute in preparing low- or non-calorie food and beverages.

Synthesis of sucralose from sucrose requires chlorination at 4-, 1′- and 6′-positions of sucrose. Two primary hydroxyl groups (1′ and 6′) and one secondary hydroxyl group (4-) need to be replaced with chlorine while the third primary hydroxyl group (6-) is unaffected. Therefore, one route to synthesize sucralose is to first protect the 6-hydroxyl group via esterification to generate sucrose-6-ester, convert the partly protected sucrose to sucralose-6-ester by selective chlorination under certain conditions, with optional esterification of all the hydroxyl groups for the purpose of purification, the protected sucralose is finally deesterified to form sucralose.

One process for the preparation of sucrose-6-ester comprises formation of a sucrose alkyl 4,6-orthoester which is hydrolized to generate a mixture of 4- and 6-monoesters of sucrose. The sucrose-4-ester is then converted to sucroses-6-ester under basic condition (U.S. Pat. No. 5,440,026).

Sucrose-6-ester can also be prepared via tin-mediated reactions. Acetic anhydride is used as esterifying agent and dibutyl tin as catalyst. These synthetic processes have certain advantages, but also have limitations.

Accordingly, there is a need in the art for a simple one-step synthesis route for sucrose-6-ester through an ester exchange reaction.

### SUMMARY OF THE INVENTION

IN one embodiment of the present invention a method for the synthesis of a sucrose-6-ester is provided, the method comprising reacting a mixture comprising sucrose, an ester and an organic solvent with a solid super acid catalyst for a period of time and at a temperature sufficient to produce sucrose 6-ester, wherein the ester comprises ethyl acetate, the organic solvent comprises DMF and wherein the solid super acid comprises SO₄²⁻—TiO₂/Al₂O₃.

In another embodiment of the present invention a method for the synthesis of a sucrose-6-ester is provided, the method comprising reacting a mixture comprising sucrose, an ester and an organic solvent with a solid super acid catalyst for a period of time and at a temperature sufficient to produce sucrose 6-ester, wherein the ester comprises ethyl acetate, the organic solvent comprises DMF and wherein the solid super acid comprises SO₄²⁻—TiO₂.

### DETAILED DESCRIPTION

The following description of the invention is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

In one embodiment of the present invention a new method for the synthesis of sucrose-6-ester is described. The catalyst used in the invention is stable, easy to recover and reusable. This embodiment can be applied to a process of making sucralose and may comprise:
(1) protection of the 6-hydroxyl group via ester-exchange reaction with an ester in the presence of a super solid acid catalyst to generate sucrose-6-ester;
(2) conversion of the partly protected sucrose to sucralose-6-ester by selective chlorination under certain conditions. This step is more fully described in U.S. patent application titled ______, Ser. No. not yet assigned;
(3) optional esterification of all the unprotected hydroxyl groups of sucralose-6-ester to form 4,1′,6′-trichloro-4,1′,6′-trideoxygalactosucrose pentaacetate (TGSPA) for the purpose of purification;
(4) de-esterification of the completely or partly protected sucralose to produce sucralose.

According to this embodiment present invention, a process for the synthesis of sucrose-6-ester from sucrose comprises reacting a mixture comprising sucrose, an ester and an organic solvent with a solid super acid catalyst for a period of time and at a temperature sufficient to produce sucrose-6-ester. The catalyst is then filtered and can be reused for the same reaction. The ester is distilled to afford a mixture comprising sucrose-6-ester and the organic solvent. If the organic solvent is one that is compatible for the chlorination reation, the obtained sucrose-6-ester solution can be used for the next step in sucralose synthesis without further purification.

The choice of organic solvents is determined by the solubility of the sucrose and the ester in the solvents, as well as the safety and toxicity considerations, especially if the sucrose-6-ester is to be used for the syntheis of sucralose, a food additive. Another consideration that should be taken into account in selecting a solvent is whether the solvent is suitable for the next step in sucralose synthesis, the chlorination reaction. The solvent is preferably a polar inorganic solvent. The polar organic solvent is preferably N,N-dimethylformamide (DMF) as DMF is a suitable solvent for the chlorination reaction. The product of the esterification reaction results in a DMF solution of sucrose-6-ester can be used directly for chlorination reaction without further purification.

The amount of the organic solvent to be used may be determined by the above-mentioned solubility considerations. When the polar solvent is DMF, it is preferably used in an amount of approximately 5 mL/g sucrose.

The ester is preferably ethyl acetate (EtOAc).

The amount of the ester to be used will be determined to facilitate the conversion of the desired sucrose-6-ester and suppress the formation of outgrowth. When the ester is EtOAc, it is preferably used in an amount of from 5 to 12 mol/mol sucrose.

The solid super acid catalyst may be selected from a group consists of one or a mixture of sulfated oxide of an element selected from those of group 3, group 4, group 5, group 6, group 7, group 8 group 9, group 10, group 11, group 12, group 13, group 14, group 15 and those of the series of lanthanides, alone or combined with each other. Examples of solid super acid catalyst include SO₄²⁻—TiO₂/Al₂O₃ , SO₄²⁻—Fe₂O₃/Al₂O₃, SO₄²⁻—ZnO/Al₂O₃, SO₄²⁻—CeO₂/Al₂O₃, SO₄²⁻—ZrO₂/Al₂O₃, SO₄²⁻—TiO₂/Al₂O₃ or SO₄²⁻—TiO₂, and the more preferable catalysts are SO₄²⁻—TiO₂/Al₂O₃ and SO₄²⁻—TiO₂.

The catalyst may be SO₄²⁻—TiO₂/Al₂O₃, Al₂O₃ infused in titanous sulfate solution and then calcinated to generate SO₄²⁻—TiO₂/Al₂O₃ solid super acid.

When the catalyst is SO₄²⁻—TiO₂, SO₄²⁻—TiO₂ solid super acid may be prepared by titanous sulfate calcinations.

In one embodiment of the invention, a one-step synthesis of sucrose-6-acetate comprises selective esterification with EtOAc at the 6-position of sucrose in the presence of solid super acid such as SO₄²⁻—TiO₂/Al₂O₃ or SO₄²⁻—TiO₂.

### EXAMPLES

### Example 1

### Synthesis of sucrose-6-acetate

Sucrose (100 g, 0.29 mol), DMF (500 mL), EtOAc (200 mL, 2.04 mol) and SO₄²⁻—TiO₂/Al₂O₃ catalyst (2 g) were stirred for 6 h at 80° C. The reaction mixture was cooled to room temperature and filtered to recover the catalyst. The filtrate was distilled to remove ethyl acetate and afford DMF solution of sucrose-6-acetate (90 g, 0.23 mol, yield 79%).

### Example 2

### Synthesis of sucralose 6-acetate

1. Thionyl chloride method

DMF (400 mL) and toluene (50 mL) were added to a DMF solution of sucrose 6-acetate (90 g, 0.23 mol) and cooled to −10° C. Thionyl chloride was added into the sucrose 6-acetate solution dropwise to maintained the temperature of the reaction below 0° C. After the addition was complete, the reaction was stirred below 5° C. for 1 h, then heated to 75-80° C. and maintained at the temperature for 1 h. Finally the reaction mixture was heated to reflux at 110-115° C. for 4 h. After the reaction was complete, the reaction was cooled by ice water. Ammonia hydroxide/methanol (1:1, approximately 500 mL) was added dropwisely to afford a solution of pH 8-9. The pH of the solution was further adjusted to 6-7 by acetic acid after stiration. Toluene was then removed by distillation at normal pressure and DMF was distilled under reduced pressure. After most of the DMF is removed, distilled water (150 mL) and ethyl acetate (900 mL) were added to the mixture and stirred for 1.5 h. The mixture was filtered and washed with ethyl acetate (200 mL). The water phase was extracted with ethyl acetate (3×300 mL). The combined organic phase was washed by brine (2×200 mL), and then concentrated to 900 mL solution under reduced pressure at a temperature below 60° C. The solution was decolorized by activated charcoal (15 g), filtered and concentrated to sucralose 6-acylate syrup (120 g) containing 60 g/0.136 mol of sucralose 6-acetate with a yield of 58%.

2. BTC Method

Sucrose 6-acetate (30 g, 008 mol) was dissolved in DMF (300 mL) and cooled to −10° C. BTC (80 g, 027 mol) was dissolved in toluene (400 mL) at a temperature below 5° C. The BTC toluene solution was cooled below 5° C. and added to the sucrose 6-acetate DMF solution slowly to maintain the reaction temperature below 0° C. The reaction mixture was stirred for 1 h after the addition is complete and heated to about 10° C., maintained at 10° C. for 2 h, then heated to 110° C. slowly. The reaction was refluxed at 110° C. for 4 h and cooled to 0° C. after the reaction was complete. Ammonia hydroxide/methanol (1:1, approximately 500 mL) was added dropwisely to afford a solution of pH 8-9. The pH of the solution was further adjusted to 6-7 by acetic acid after stiration. Toluene was then removed by distillation at room temperature and DMF was distilled under reduced pressure. When most DMF is removed, distilled water (100 mL) and ethyl acetate (500 mL) were added to the mixture and stirred for 1 h. The mixture was filtered and washed with ethyl acetate (150 mL). The aqueous layer was extracted with ethyl acetate (3×200 mL). The combined organic phase was washed by brine (2×100 mL), and then concentrated to 400 mL solution under reduced pressure at a temperature below 60° C. The solution was decolorized by activated charcoal (10 g), filtered and concentrated to sucralose 6-acylate syrup (40 g) containing 22 g/0.05 mol of sucralose 6-acetate with a yield of 62%.

### Example 3

### Preparation of TGSPA

Acetate syrup (40 g, contained 22 g/0.05 mol of sucralose 6-acetate), which was prepared in example 2, was added to acetic anhydride (100 mL, 1.05 mol). Pyridine (2 mL) was added thereto and the reaction was stirred at 50° C. for 3 h. The reaction mixture was then cooled to 20° C. Methanol (60 mL) was added dropwise to maintain the reaction temperature below 50° C. The mixture was distilled at a temperature below 60° C. under reduced pressure to get TGSPA syrup. The obtained syrup was dissolved in toluene (300 mL) and washed by brine (50 mL). The combined organic phase was distilled to TGSPA concentrated syrup under reduced pressure at a temperature below 60° C. The obtained syrup was dissolved in toluene (40 mL) at 70° C. and cooled to room temperature for recrystallization. The crystals were filtered recrystallized two more times to yield about 26 g pure TGSPA (0.42 mol, 85% yield).

### Example 4

### Preparation of sucralose

TGSPA (10 g, 0.016 mol) was dissolved in methanol (100 mL) and cooled to 15° C. 20% Sodium methoxide/methanol solution (4 g, 0.015 mol) was added thereto and stirred for 5 h at room temperature. After the reaction was complete it was neutralized and filtered by hydrogen strong acid ion exchange resin, which was consequently washed by methanol (2×50 mL). The filtrate was distilled to soft foam under reduced pressure below 30° C. The foam was dissolved in distilled water (100 mL), and the solution was extracted by ethyl acetate (50 mL). The aqueous phase was then decolored with activated charcoal (0.5 g), filtered to remove the activated charcoal and washed with distilled water (2×300 mL). The filtrate was concentrated to syrup by distillation under reduced pressure at room temperature. Distilled water (8 mL) was added to dissolve the syrup at 80° C. After the solution was cooled to below 20° C., crystal seeds were added to the solution. The formed crystals were filtered and washed by small amount of cold water, dried, then dried in crystallizing dish under reduced pressure at 45-50° C. to produce sucralose (5 g, 0.013 mol, yield 83%).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

What is claimed is:

1. A method for the synthesis of a sucrose-6-ester comprising:
providing a mixture of sucrose, an ester, and an organic solvent, and
reacting the mixture with a solid super acid catalyst for a period of time and at a temperature sufficient to produce sucrose-6-ester.

2. The method of claim 1, wherein the ester comprises ethyl acetate.

3. The method of claim 1, wherein the organic solvent comprises DMF.

4. The method of claim 1, wherein the solid super acid is selected from a group consisting of one or more of sulfated oxide of an element selected from those of group 3, group 4, group 5, group 6, group 7, group 8 group 9, group 10, group 11, group 12, group 13, group 14, group 15 and those of the series of lanthanides, alone or combined with each other.

5. The method of claim 4, wherein the solid super acid catalyst comprises SO₄²⁻—TiO₂/Al₂O₃.

6. The method of claim 4, wherein the solid super acid catalyst comprises SO₄²⁻—TiO₂.

7. The method of claim 5 wherein Al₂O₃ is infused in titanous sulfate solution and then calcinated to generate SO₄^{2-l —TiO}₂/Al₂O₃ solid super acid.

8. The method of claim 6 wherein SO₄²⁻—TiO₂ solid super acid is prepared by titanous sulfate calcinations.

9. The method of claim 2, wherein the mole equivalent (ME) of ethyl acetate:sucrose is in a range from 5:1 to 12:1.

10. A method for the preparation of sucralose comprising preparation of sucrose 6-ester according to the method of claim 1.

11. A method for the preparation of sucralose, comprising preparing a sucrose 6-ester in one step esterification, reacting the sucrose 6-ester with a chlorinating agent capable of selectively chlorinating the 4-, 1′- and 6′ positions, optionally pre-esterifying the sucralose 6-ester so formed, de-esterifying the sucralose ester, and recovering sucralose, wherein the sucrose 6-ester is prepared according to the process of claim 1.

12. The method of claim 11 wherein the chlorinating agent comprises BTC.

13. The method of claim 10, wherein the ester comprises ethyl acetate.

14. The method of claim 10, wherein the organic solvent comprises DMF.

15. The method of claim 10, wherein the solid super acid catalyst comprises SO₄²⁻—TiO₂/Al₂O₃.

16. The method of claim 10, wherein the solid super acid catalyst comprises SO₄²⁻—TiO₂.

17. The method of claim 14 wherein Al₂O₃ was infused in titanous sulfate solution and then calcinated to generate SO₄²⁻—TiO₂/Al₂O₃ solid super acid.

18. The method of claim 15 wherein SO₄²⁻—TiO₂ solid super acid was prepared by titanous sulfate calcinations.

19. The method of claim 10 comprising chlorinating sucrose 6-acetate with BTC, wherein the sucrose 6-acetate is dissolved in an organic solvent and wherein the BTC is dissolved in one or several organic solvent to prepare a Vilsmeier reagent of BTC solution, which was added to the solution of sucrose 6-acetate for chlorination reaction.

20. The method of claim 17 wherein the organic solvent is selected from a group consisting of DMF, cyclohexane, toluene, dichloethane, chloroform, carbon tetrachloride and ethyl acetate.

21. The process of claim 10, wherein the chlorination reaction proceeds at normal pressure or reduced pressure.

22. A method for the synthesis of a sucrose 6-ester, the method comprising
reacting a mixture comprising sucrose, an ester and an organic solvent with a solid super acid catalyst for a period of time and at a temperature sufficient to produce sucrose 6-ester,
wherein the ester comprises ethyl acetate, the organic solvent comprises DMF and wherein the solid super acid comprises SO₄²⁻—TiO₂/Al₂O₃.

23. A method for the synthesis of a sucrose 6-ester, the method comprising
reacting a mixture comprising sucrose, an ester and an organic solvent with a solid super acid catalyst for a period of time and at a temperature sufficient to produce sucrose 6-ester,
wherein the ester comprises ethyl acetate, the organic solvent comprises DMF and wherein the solid super acid comprises SO₄²⁻—TiO₂.
